(19) United States
(12) Patent Application Publication
(10) Pub. No.: US 2015/0306273 A1
(43) Pub. Date: Oct. 29, 2015

(54) MEDICAL SEALANT COMPOSITION AND METHOD OF USING SAME

(71) Applicant: 3M INNOVATIVE PROPERTIES COMPANY, Saint Paul, MN (US)

(72) Inventors: NAIMUL KARIM, MAPLEWOOD, MN (US); HAE-SEUNG LEE, WOODBURY, MN (US)

(73) Assignee: 3M INNOVATIVE PROPERTIES COMPANY, Saint Paul, MN (US)

(21) Appl. No.: 14/652,457

(22) PCT Filed: Dec. 13, 2013
(86) PCT No.: PCT/US2013/074855

§ 371 Date: Jun. 16, 2015

Related U.S. Application Data
(60) Provisional application No. 61/738,521, filed on Dec. 18, 2012.

(51) Int. Cl. A61L 24/04; A61M 1/00; A61F 13/00

(52) U.S. Cl. CPC A61L 24/043; A61F 13/00068; A61M 1/0088; A61M 2209/088; A61M 2202/0014; A61B 17/00491

(57) ABSTRACT

A medical sealant composition and method for coupling the medical article to skin using the medical sealant composition. The medical sealant composition can include an unsaturated rubber hydrocarbon having at least one hydrosilyation-crosslinkable functional group and a crosslinking agent having at least one SiH group per molecule. The medical sealant composition can cure at 35 degrees C. in less than 20 minutes. The method can include applying the composition to one or both of a medical article and skin when the composition is in an uncured state; applying the medical article to the skin; and allowing the composition to cure to form a sealant between the medical article and the skin.

### FIELD

The present disclosure generally relates to a medical sealant composition and methods for coupling a medical article to skin using the medical sealant composition.

### BACKGROUND

A wide variety of medical articles need to be coupled to skin in use. In some cases, it can be important to achieve a good seal between the medical article and the skin. For example, negative pressure wound therapy (NPWT) employs controlled vacuum to promote healing in acute or chronic wounds. Achieving a good seal in NPWT treatment can be difficult and/or time-consuming, for example, when a three-dimensional body part is covered by a substantially flat medical article.

### SUMMARY

The present disclosure relates to a medical sealant composition that can be used to couple a medical article to skin and methods for coupling the medical article to skin using the medical sealant composition. One feature and advantage of the medical sealant of the present disclosure is that it can provide a simple, robust and effective solution for coupling medical articles to skin. As a result, in some embodiments, the medical sealant of the present disclosure can provide a better approach for creating and maintaining a vacuum under an NPWT dressing, while minimizing leakage of the vacuum and wound exudates.

Some aspects of the present disclosure provide a medical sealant composition. The medical sealant composition can include an unsaturated rubber hydrocarbon having at least one hydrosilylation-crosslinkable functional group and a crosslinking agent having at least one SiH group per molecule. The medical sealant composition can cure at 35 degrees C. in less than 20 minutes.

Some aspects of the present disclosure provide a method for coupling a medical article to skin. The method can include providing a medical article; providing a composition comprising an unsaturated rubber hydrocarbon having at least one hydrosilylation-crosslinkable functional group and a crosslinking agent having on the average at least one SiH group per molecule; applying the composition to one or both of the medical article and skin when the composition is in an uncured state; applying the medical article to the skin; and allowing the composition to cure to form a sealant between the medical article and the skin.

Other features and aspects of the present disclosure will become apparent by consideration of the detailed description and accompanying drawings.

### BRIEF DESCRIPTION OF THE DRAWINGS

FIG. 1 is a schematic perspective view of a negative pressure wound therapy system comprising a medical sealant according to one embodiment of the present disclosure.

FIG. 2 is a schematic partial cross-sectional view of the negative pressure wound therapy system of FIG. 1.

FIG. 3 is a schematic cross-sectional view of an experimental negative pressure wound therapy system used in the examples.

FIG. 4 is a bottom plan view of the experimental negative pressure wound therapy system of FIG. 3.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “coupled” and variations thereof are used broadly and encompass both direct and indirect couplings. Further, “coupled” is not restricted to physical or mechanical couplings. It is to be understood that other embodiments may be utilized, and structural or logical changes may be made without departing from the scope of the present disclosure.

The present disclosure generally relates to a medical sealant composition and methods for coupling a medical article to skin using the medical sealant composition. Particularly, the medical sealant composition of the present disclosure can be curable, or configured to cure, quickly, generally within 20 minutes at 35 degrees C. When the sealant is no longer needed, the cured composition can be easily removed without leaving residue. As a result, the removal of the cured composition can be clean and painless. The composition can be provided to couple (i.e., fluidly seal) a medical article to skin. When the composition is provided, the composition can be applied to one or both of the medical article and skin in a first uncured state and can cure to form a sealant between the medical article and the skin. The composition of the present disclosure therefore provides a simple, robust and effective solution for coupling medical articles to skin, and particularly, for providing a reliable seal between medical articles and skin.

In some embodiments, the method can include providing a medical article and a composition; applying the composition to one or both of the medical article and skin when the composition is in an uncured state; applying the medical article to the skin; and allowing the composition to cure to form a sealant between the medical article and the skin. In some embodiments, applying the composition comprises dispensing the composition from a dual-cartridge automix delivery system.

In some embodiments, the medical sealant composition can include an unsaturated rubber hydrocarbon having at least one hydrosilylation-crosslinkable functional group and a crosslinking agent having on average at least one SiH group per molecule.

In some embodiments, the medical sealant composition of the present disclosure can be used as a sealant for a negative pressure wound therapy system (NPWT), or reduced-pressure wound therapy. NPWT has been used to promote healing across a wide range of wound types. NPWT generally uses a controlled vacuum to promote healing in acute or chronic wounds. NPWT involves the application of a vacuum to the wound bed, and is generally attained by covering the wound with an adhesive coated dressing, to which a vacuum pump (or other reduced-pressure source) is attached. The dressing can prevent leakage of the vacuum and wound exudates. Achieving a good seal between the dressing and the skin can be difficult and time-consuming. One reason is the tendency for radial folds and creases to be created when a three-dimensional body part is covered by a flat dressing or sheet. These folds and creases can create channels for air and exudates. As a result, clinicians can spend a lot of time cutting small pieces of additional dressing material and patching up the channels. Even after a seal has been attained, leaks can develop due to stretching and flexing of body parts.

The compositions of the present disclosure can wet a rough surface (e.g. skin) easily and can cure within minutes to a soft and compliant solid. When the soft, cured composition is no longer needed, it can be painlessly removed without leaving residue. The compositions of the present disclosure can act as a sealant, including a sealant used for NPWT. The compositions of the present disclosure can provide better sealing to seal the leakage occurring in NPWT treatment, and thus can reduce power consumption and extend battery life of an NPWT system, which can be especially important for portable devices. As a result, the compositions of the present disclosure also facilitate the use of smaller portable pumps in the NPWT system. Therefore, the compositions of the present disclosure can provide a simple, robust and effective solution for creating and maintaining a vacuum under an NPWT dressing, while minimizing leakage of the vacuum and wound exudates.

In some embodiments, a component of a negative pressure wound therapy system (e.g., a sealing member configured to cover a wound and provide connection to a reduced-pressure source) can be provided as the medical article, the composition can be applied to one or both of the component and the skin, and the component can be applied to the skin after applying the composition. By way of example, in some embodiments, the component (e.g., the sealing member) can include a dressing, a drape, or the like, or combinations thereof.

In some embodiments, compositions of the present disclosure can cure at 35 degrees C. (i.e., approximately body temperature) in less than 20 minutes. In some embodiments, the composition of the present disclosure can cure at 35 degrees C. in less than 15 minutes. In some embodiments, the composition of the present disclosure can cure at 35 degrees C. in less than 10 minutes. In some embodiments, the composition of the present disclosure can cure at 35 degrees C. in less than 5 minutes.

The term “cured” generally refers to a state when the composition demonstrates elasticity (e.g., tactilely) and leaves no residue (e.g., on a fingertip when touched). For example, the compositions can be considered to cure when the composition becomes a viscoelastic, non-flowing solid with tackiness and resilience. At this stage, typical cured compositions show good physical integrity and leave very limited residue.

In some embodiments, compositions of the present disclosure have a first (uncured) state having a viscosity of at least 15,000 cP. In some embodiments, compositions of the present disclosure have a first state having a viscosity of at least 20,000 cP. In some embodiments, compositions of the present disclosure have a first state having a viscosity of at least 45,000 cP. In some embodiments, compositions of the present disclosure have a first state having a viscosity of no greater than 1,000,000 cP. Such viscosity ranges, for example, can allow the composition to easily wet out a rough surface (e.g., skin) when the composition is in its first state, without being too wetting or runny. At viscosities of greater than 1,000,000 cP, the composition can begin to become too viscous and/or not easily pumpable or dispensable.

In some embodiments, compositions of the present disclosure form a second (cured) state having a shore-hardness ranging from about 10 to about 50, after curing. In some embodiments, compositions of the present disclosure form a second state having a shore-hardness ranging from about 15 to about 40, after curing. In some embodiments, compositions of the present disclosure form a second state having a shore-hardness ranging from about 15 to about 35, after curing. In some embodiments, compositions of the present disclosure form a second state having a shore-hardness ranging from about 20 to about 30, after curing. Such hardness ranges, for example, can provide sufficient structural integrity while also allowing the composition to be soft and compliant, e.g., to function to seal a medical article to skin. The cured compositions maintain a certain amount of tack and can act as a sealant.

In some embodiments, the unsaturated rubber hydrocarbon can include ethylenepropylene-diene rubber (EPDM). In some embodiments, the EPDM can include a norbornene derivative having a vinyl group. In some embodiments, the unsaturated rubber hydrocarbon can be selected from 5-vinyl-2-norbornene, isobutylene-isoprenedivinylbenzene rubber (IIR terpolymer), isobutyleneisoprene rubber (IIR), butadiene rubber (BR), styrenebutadiene rubber (SBR), styrene-isoprene rubber (SIR), isoprene-butadiene rubber (IBR), isoprene rubber (IR), acrylonitrile-butadiene rubber (NBR), chloroprene rubber (CR), acrylate rubber (ACM) or partially hydrogenated rubber from butadiene rubber (BR), styrenebutadiene rubber (SBR), isoprene-butadiene rubber (IBR), isoprene rubber (IR), acrylonitrile-butadiene rubber (NBR), polyisobutylene rubber (PIB) having two vinyl groups, functionalized rubber (e.g., perfluoropolyether rubber functionalized with maleic anhydride or derivatives thereof or with vinyl groups), or combinations thereof.

In some embodiments, the unsaturated rubber hydrocarbon can include ethylene-propylene-diene rubber (EPDM) with a vinyl group in the diene, polyisobutylene (PIB) having two terminal vinyl groups, acrylonitrile-butadiene rubber (NBR) or acrylate rubber (ACM).

In some embodiments, the unsaturated rubber hydrocarbon can include polyisoprene according to the following general formula (I):

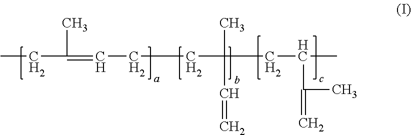

In some embodiments, the polyisoprene has a molecular weight ranging from about 5,000 to about 100,000. A weight average molecular weight of at least 5,000 can be useful in diminishing curing time, e.g., to ensure that the curing time at 35 degrees C. is less than 20 minutes. On the other hand, the weight average molecular weight of the polymer is generally not more than 100,000 or the polymer begins to become a solid and is not easily pumpable. In some embodiments employing polyisoprene, the polyisoprene has a molecular weight ranging from about 10,000 to about 90,000. In some embodiments employing polyisoprene, the polyisoprene has a molecular weight ranging from about 20,000 to about 80,000.

The crosslinking agents that can be used in the present discourse have at least 1 hydrosilyl group per molecule. Crosslinking agents of this type are described in detail in U.S. Pat. No. 6,087,456, which is incorporated herein by reference.

In some embodiments, the crosslinking agent can include a compound of formula (III) comprising SiH:

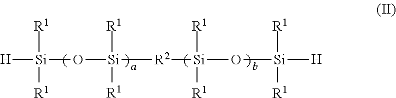

wherein R¹ stands for a saturated hydrocarbon group or an aromatic hydrocarbon group which is monovalent, has 1 to 10 carbon atoms, and is substituted or unsubstituted, wherein “a” stands for integer values from 0 to 20 and “b” stands for integer values from 0 to 20, and R² stands for a divalent organic group having 1 to 30 carbon atoms or oxygen atoms.

In some embodiments, the crosslinking agent can include a compound of formula (III) comprising SiH:

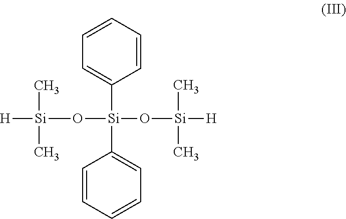 [SMILES: C[SiH](C)O[Si](O[SiH](C)C)(c1ccccc1)c1ccccc1]

In some embodiments, the crosslinking agent can include a compound of formula (IV) comprising SiH:

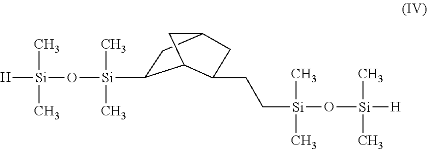 [SMILES: C[SiH](C)O[Si](C)(C)CCC1CC2CC1C([Si](C)(C)O[SiH](C)C)C2]

In some embodiments, the crosslinking agent can include a compound of formula (V) comprising SiH:

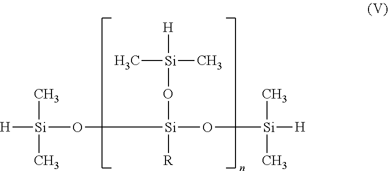

wherein n represents an integer from 1 to about 3, wherein R represents an alkyl group containing from 1 to 4 carbon atoms, a phenyl group, or a hydrosilyl group.

In some embodiments, the crosslinking agent can be selected from poly(dimethylsiloxane-comethylhydrosiloxane), tris(dimethylsilyloxy)phenyl silane, bis(dimethylsilyloxy)diphenylsilane, polyphenyl(dimethylhydrosiloxy)siloxane, methylhydrosiloxane-phenylmethylsiloxane copolymer, methylhydrosiloxane-alkylmethylsiloxane copolymer, polyalkylhydrosiloxane, methylhydrosiloxane-diphenylsiloxanealkylmethylsiloxane copolymer and/or from polyphenylmethylsiloxane-methylhydrosiloxane.

In some embodiments, the crosslinking agent can be a tetrakis(dialkyl siloxy) silane or a tris(dialkyl siloxy)alkyl silane. In other embodiments, the crosslinking agent can be a branched silane coupling agent such as tetrakis(dimethyl siloxy) silane, tris(dimethyl siloxy) methyl silane, and tris(dimethyl siloxy) phenyl silane.

In some embodiments, the crosslinking agent can be poly(dimethylsiloxane-comethylhydrosiloxane), tris(dimethylsilyloxy)phenylsilane or bis(dimethylsilyloxy)diphenylsilane.

In some embodiments, the crosslinking agent can be 1,3,5,7-tetramethylcyclotetrasiloxane. In some embodiments, the crosslinking agent can be 1,1,4,4-tetramethyl-disilabutane.

In some embodiments, compositions of the present disclosure can further comprise a polymer diluent. The polymer diluent can function to reduce the density of the cross-linking agent so as to prevent over cross-linking of the composition and to maintain a desired flexibility of the composition. In some embodiments, the addition of diluents can reduce the viscosity of the compositions, which can enable easier application. In some embodiments, the polymer diluent can include an unreactive rubber, mineral oil, or a combination thereof. In some embodiments, the polymer diluent is polyisobutylene.

In some embodiments, compositions of the present disclosure can further comprise a catalyst. A wide variety of catalysts can be used in the compositions of the present disclosure. Some representative examples of suitable catalysts include, but are not limited to, chloroplatinic acid, elemental platinum, solid platinum supported on a carrier (such as alumina, silica or carbon black), platinum-vinylsiloxane complexes {for instance: Ptn(ViMe₂SiOSiMeVi)n and Pt[(MeViSiO)₄]m}, platinum-phosphine complexes {for example: Pt(PPh₃)₄ and Pt(PBu₃)₄}, platinum-phosphite complexes {for instance: Pt[P(OPh)_{3]4} and Pt[P(OBu)_{3]4}}, or combinations thereof, where Me rep-resents methyl, Bu represents butyl, Vi represents vinyl and Ph represents phenyl, and n and m represent integers. The platinum-hydrocarbon complex described in the specification of U.S. Pat. No. 3,159,601 and U.S. Pat. No. 3,159,662, and the platinum-alcoholate catalyst described in the specification of U.S. Pat. No. 3,220,972 can also be used. U.S. Pat. No. 3,159,601, U.S. Pat. No. 3,159,662, and U.S. Pat. No. 3,220,972 are each incorporated herein by reference.

In some embodiments, the catalyst can be selected from platinum(0)-1,3-divinyl-1,1,3,3-tetramethyldisiloxane complex, hexachloroplatinic acid, dichloro(1,5-cyclooctadiene)platinum(II), dichloro(dicyclopentadienyl)platinum(II), tetrakis(triphenylphosphine)platinum(0), chloro(1,5-cyclooctadiene)rhodium(I) dimer, chlorotris(triphenylphosphine)rhodium(I) and/or dichloro(1,5-cyclooctadiene)palladium(II), optionally in combination with a kinetic regulator selected from dialkyl maleate, in particular dimethyl maleate, 1,3,5,7-tetramethyl-1,3,5,7-tetravinylcyclosiloxane, 2-methyl-3-butyn-2-ol and/or 1-ethynylcyclohexanol.

In some embodiments, the catalyst can be a platinum-divinyltetramethyldisiloxane complex.

In some embodiments, compositions of the present disclosure can be at least a two-part system having, at least, a first part comprising the unsaturated rubber hydrocarbon and a second part comprising the unsaturated rubber hydrocarbon and the crosslinking agent. In some embodiments, the unsaturated rubber hydrocarbon of the first part may be different from the unsaturated rubber hydrocarbon of the second part. In some embodiments, the first part can further comprise a catalyst. In some embodiments, the first part and the second part of the two-part system are kept separate prior to use.

In the case of the two-part system, the crosslinking agent and the catalyst are added separately from one another, i.e., in two systems, cartridges or containers, each mixed first with the unsaturated rubber hydrocarbon until achieving a homogeneous distribution before the two systems, i.e., the mixture with the crosslinking agent and the mixture with the catalyst are combined and all the components are mixed together. The two-part system has the advantage that the two mixtures in which the crosslinking agent and the catalyst are separate from one another are stable for a longer period of time than a mixture that contains both the crosslinking agent and the hydrosilylation catalyst system. As a result, the two-part system has a longer shelf life.

In some embodiments, the composition of the present disclosure can be a multiple-part system having more than two parts, each part comprising at least one component of the composition of the present disclosure.

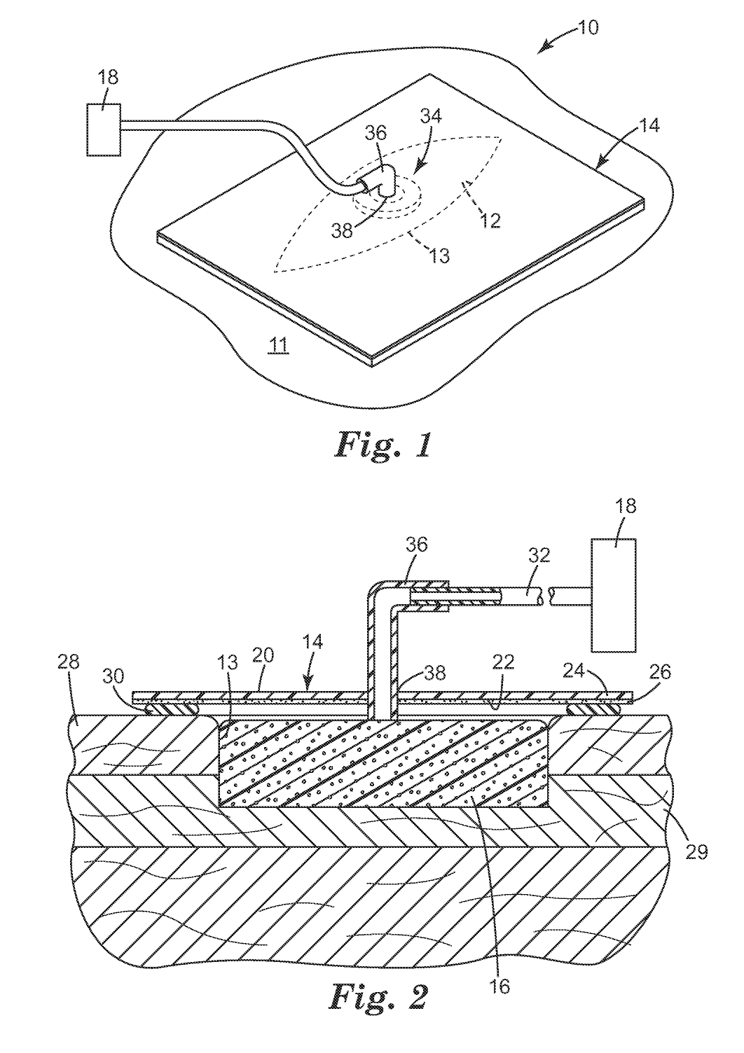

FIGS. 1-2 illustrate a negative or reduced pressure wound therapy system 10 according to one embodiment of the present disclosure. As shown in FIG. 1, in some embodiments, the negative pressure wound therapy system 10 can be applied to a patient's skin 11 comprising a wound 12. FIG. 2 schematically illustrates various layers of the patient's skin 11, including an epidermis 28, and a dermis 29.

As shown in FIGS. 1-2, the negative pressure wound therapy system 10 can include a sealing member 14, a manifold 16, and a negative or reduced pressure source 18.

The sealing member 14 can be formed from a flexible sheet. The sealing member 14 includes a first surface 20 and a second, tissue-facing surface 22. The sealing member 14 can be sized so that the sealing member 14 overlaps the wound 12 in such a manner that a drape extension 24 extends beyond a peripheral edge 13 of the wound 12.

The sealing member 14 may form, or aid in forming, a fluid seal over the wound 12. The sealing member 14 may be formed from any material that provides a fluid seal. As used herein, “fluid seal,” or “seal,” generally refers to a seal adequate to maintain reduced pressure at a desired site, e.g., a tissue site, given the particular reduced-pressure source involved. The sealing member may, for example, be an impermeable or semi-permeable, elastomeric material. “Elastomeric” generally refers to having the properties of an elastomer. Elastomeric generally refers to a polymeric material that has rubber-like properties. More specifically, most elastomers have ultimate elongations greater than 100% and a significant amount of resilience. The resilience of a material refers to the material's ability to recover from an elastic deformation. Examples of elastomers may include, but are not limited to, natural rubbers, polyisoprene, styrene butadiene rubber, chloroprene rubber, polybutadiene, nitrile rubber, butyl rubber, ethylene propylene rubber, ethylene propylenediene monomer, chlorosulfonated polyethylene, polysulfide rubber, polyurethane, EVA film, co-polyester, and silicones.

Specific examples of sealing member materials include, but are not limited to, a silicone drape or dressing; a drape or dressing, available under the trade designation 3M® TEGADERM® from 3M Company, St. Paul, Minn.; an acrylic drape or dressing such as one available from Avery Dennison; an incise drape or dressing; or combinations thereof.

In some embodiments, an attachment member 26 may be used to additionally couple the sealing member 14 to a patient's epidermis 28 or another layer, such as a gasket or additional sealing member. The attachment member 26, if employed, can be operable to removably couple the sealing member 14 to a patient's epidermis 28. As mentioned above, the term “coupled” can include direct or indirect couplings. The term “coupled” can also encompass two or more components that are continuous with one another by virtue of each of the components being formed from the same piece of material, i.e., integral. Also, in some embodiments, the term “coupled” can include chemical coupling means, such as via a chemical bond; mechanical coupling means; thermal coupling means; electrical coupling means, or a combination thereof.

The attachment member 26 may be any material suitable to help couple the sealing member 14 to a patient's epidermis 28. For example, the attachment member 26 may be a pressure-sensitive adhesive (PSA), a heat-activated adhesive, a sealing tape, a double-sided sealing tape, a paste, a hydrocolloid, a hydrogel, hooks, sutures, other sealing devices or elements, or a combination thereof. By way of example only, the attachment member 26 shown in FIG. 2 is a PSA.

In some embodiments, a layer of sealant bead 30 comprising the medical sealant composition of the present disclosure can be used to fluidly seal (e.g., hermetically) the sealing member 14 (and/or the attachment member 26) against the patient's epidermis 28. The sealing member 14 (and/or attachment member 26) and the sealant bead 30 work together to form a fluid seal over the patient's epidermis 28.

As shown in FIG. 2, in some embodiments, the manifold 16 can be disposed proximate or within the wound 12. The term “manifold” as used herein generally refers to a substance or structure that is provided to assist in applying negative or reduced pressure to, delivering fluids to, or removing fluids from a tissue site or wound 12.

The manifold 16 generally includes a plurality of flow channels or pathways that distribute fluids provided to and removed from the tissue site or wound 12 around the manifold 16. In some embodiments, the flow channels or pathways are interconnected to improve distribution of fluids provided or removed from the wound 12. The manifold 16 may be a biocompatible material that is capable of being placed in contact with the wound 12 and distributing negative or reduced pressure to the wound 12.

Examples of manifolds 16 may include, for example, but are not limited to, devices that have structural elements arranged to form flow channels, such as, for example, cellular foam, open-cell foam, porous tissue collections, liquids, gels, foams that include, or cure to include, flow channels, or combinations thereof. The manifold 16 may be porous and may be made from foam, gauze, felted mat, or any other material suited to a particular biological application. In some embodiments, the manifold 16 can be a porous foam and include a plurality of interconnected cells or pores that act as flow channels. The porous foam may be a polyurethane, open-cell, reticulated foam, such as V.A.C.® GranuFoam® material manufactured by Kinetic Concepts, Incorporated of San Antonio, Tex. Other embodiments may include closed-cell foams. In some situations, the manifold 16 may also be used to distribute fluids such as medications, antibacterials, growth factors, and various solutions to the wound 12. Other layers may be included in or on the manifold 16, such as absorptive materials, wicking materials, hydrophobic materials, and hydrophilic materials.

With continued reference to FIGS. 1 and 2, the reduced pressure supplied by the negative or reduced-pressure source 18 can be delivered through a conduit 32 to a reduced-pressure interface 34, which, in some embodiments, can include an elbow port 36. The reduced-pressure interface 34, e.g., a connector, can be disposed proximate the manifold 16 and can extend through an aperture 38 in the sealing member 14. In some embodiments, the port 36 can be a TRAC® technology port available from Kinetic Concepts, Inc. of San Antonio, Tex. The reduced-pressure interface 34 allows the reduced pressure to be delivered to the sealing member 14 and realized within an interior portion of sealing member 14 as well as the manifold 16. In this illustrative embodiment, the port 36 extends through the sealing member 14 to the manifold 16.

The negative pressure wound therapy system 10 of FIGS. 1 and 2 is shown by way of example only for the purposes of illustration and to demonstrate one potential use or application of the medical sealant composition of the present disclosure. However, it should be understood that the medical sealant composition of the present disclosure can be applied to different negative pressure wound therapy systems or other medical articles systems without departing from the spirit and scope of the present disclosure.

The following embodiments are intended to be illustrative of the present disclosure and not limiting.

### EMBODIMENTS

Embodiment 1 is a medical sealant composition comprising:
an unsaturated rubber hydrocarbon having at least one hydrosilylation-crosslinkable functional group and a crosslinking agent having at least one SiH group per molecule, wherein the composition is curable at 35 degrees C. in less than 20 minutes.

Embodiment 2 is the medical sealant composition of embodiment 1, wherein the composition is curable at 35 degrees C. in less than 15 minutes.

Embodiment 3 is the medical sealant composition of embodiment 1 or 2, wherein the composition is curable at 35 degrees C. in less than 10 minutes.

Embodiment 4 is a negative pressure wound therapy system comprising the medical sealant composition of any preceding embodiment.

Embodiment 5 is a method for coupling a medical article to skin, the method comprising: providing a medical article;
providing a composition comprising an unsaturated rubber hydrocarbon having at least one hydrosilylation-crosslinkable functional group and a crosslinking agent having on the average at least one SiH group per molecule;
applying the composition to one or both of the medical article and skin when the composition is in an uncured state;
applying the medical article to the skin; and allowing the composition to cure to form a sealant between the medical article and the skin.

Embodiment 6 is the method of embodiment 5, wherein applying the sealant includes dispensing the sealant from a dual-cartridge automix delivery system

Embodiment 7 is the method of embodiment 5 or 6, wherein the medical article is a component of negative pressure wound therapy system; wherein providing a medical article includes providing the component; wherein applying the composition to one or both of the medical article and skin includes applying the composition to one or both of the component and the skin; and wherein applying the medical article to the skin after applying the composition includes applying the component to the skin.

Embodiment 8 is the medical sealant composition of any of embodiments 1-4 or the method of any of embodiments 5-7, wherein the composition has a first (uncured) state having a viscosity of at least 15,000 cP.

Embodiment 9 is the medical sealant composition of any of embodiments 1-4 and 8 or the method of any of embodiments 5-8, wherein the composition has a first state having a viscosity of at least 20,000 cP.

Embodiment 10 is the medical sealant composition of any of embodiments 1-4 and 8-9 or the method of any of embodiments 5-9, wherein the composition has a first state having a viscosity of at least 45,000 cP.

Embodiment 11 is the medical sealant composition of any of embodiments 1-4 and 8-10 or the method of any of embodiments 5-10, wherein the composition forms a second state having a shore-hardness ranging from about 10 to about 50, after curing.

Embodiment 12 is the medical sealant composition of any of embodiments 1-4 and 8-11 or the method of any of embodiments 5-11, wherein the composition forms a second state having a shore-hardness ranging from about 15 to about 40, after curing.

Embodiment 13 is the medical sealant composition of any of embodiments 1-4 and 8-12 or the method of any of embodiments 5-12, wherein the composition forms a second state having a shore-hardness ranging from about 15 to about 35, after curing.

Embodiment 14 is the medical sealant composition of any of embodiments 1-4 and 8-13 or the method of any of embodiments 5-13, wherein the unsaturated rubber hydrocarbon comprises polyisoprene.

Embodiment 15 is the medical sealant composition of or the method of embodiment 14, wherein the polyisoprene has a molecular weight ranging from about 10000 to about 90000.

Embodiment 16 is the medical sealant composition of any of embodiments 1-4 and 8-15 or the method of any of embodiments 5-15, wherein the composition further comprises a polymer diluent.

Embodiment 17 is The medical sealant composition of or the method of embodiment 16, wherein the polymer diluents includes an unreactive rubber, mineral oil, or a combination thereof.

Embodiment 18 is the medical sealant composition of or the method of embodiment 16 or 17, wherein the polymer diluent comprises polyisobutylene.

Embodiment 19 is the medical sealant composition of any of embodiments 1-4 and 8-18 or the method of any of embodiments 5-18, wherein the composition further comprises a catalyst.

Embodiment 20 is the medical sealant composition of any of embodiments 1-4 and 8-19 or the method of any of embodiments 5-19, wherein the composition is a two-part system comprising a first part comprising the unsaturated rubber hydrocarbon and a second part comprising the unsaturated rubber hydrocarbon and the crosslinking agent.

Embodiment 21 is the medical sealant composition of or the method of embodiment 20, wherein the first part further comprises a catalyst.

Embodiment 22 is the medical sealant composition of or the method of embodiment 20 or 21, wherein the first part and the second part of the two-part system are kept separate prior to use.

The following working examples are intended to be illustrative of the present disclosure and not limiting.

### EXAMPLES

### Materials

Materials utilized for the examples are shown in Table 1.

TABLE 1
Materials List

| Compound | Description | Source |
|---|---|---|
| LIR-30 | Polyisoprene, MW 28,000 | Kuraray America, Inc, |
| | | Pasadena, TX |
| LIR-50 | Polyisoprene, MW 54,000 | Kuraray America, Inc, |
| | | Pasadena, TX |
| Diluent | Polyisobutylene, | BASF, Florham Part, NJ |
| | Glissopal ™ 1000 | |
| Catalyst | Karstedt's catalyst, Pt- | Gelest, Inc., Morrisville, PA |
| | divinyltetramethyldisiloxane | |
| TMCTS | 1,3,5,7- | Gelest, Inc., Morrisville, PA |
| | tetramethylcyclotetrasiloxane | |
| Ricon 130 | Polybutadiene, MW 2,500 | Cray Valley, Exton, PA |
| Ricon 131 | Polybutadiene, MW 4,500 | Cray Valley, Exton, PA |
| Ricon 134 | Polybutadiene, MW 8,000 | Cray Valley, Exton, PA |
| LBR 307 | Polybutadiene, MW 8,000 | Kuraray America, Inc, |
| | | Pasadena, TX |
| LBR 305 | Polybutadiene, MW 25,000 | Kuraray America Inc, |
| | | Pasadena, TX |

### Test Methods

### Cure

Samples were placed in a 35° C. oven to cure. Samples were removed every 5 minutes and visually and tactilely assessed. The sample was lightly touched and the elasticity was observed as well as the amount of material which remained on the fingertip. When the sample demonstrated elasticity and no material remained on the fingertip, the sample was determined to be fully cured. Cure time was measured in minutes or hours.

### Hardness

Hardness (Shore A) of cured sealant was measured with a type A durometer (model 306L, PCT™ Instruments, Los Angeles, Calif.). All measurements were conducted three days post cure at room temperature.

### Tack

Tack was evaluated three days post cure at room temperature by lightly touching the sample. Tack was assigned a low, medium, or high rating.

### Viscosity

Viscosity of each formulation (without added cross-linker and catalyst) was measured with a Brookfield Viscometer (model DV-II+ PRO, Middleboro, Mass.). All measurements were conducted at 23° C. with an LV-3 spindle at 1-5 rpm.

### Seal

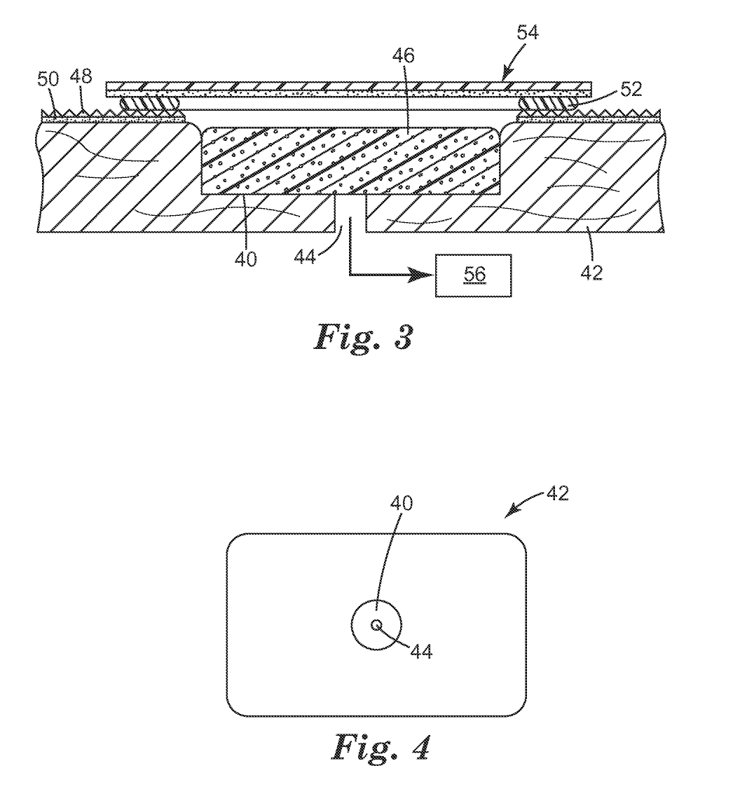

Vacuum seal testing was performed with the experimental negative pressure wound therapy system shown in FIGS. 3-4. As shown in FIG. 4, a simulated wound bed 40 was created by removing a 3.81 cm diameter, 1.91 cm deep portion of a polycarbonate block 42, which simulated a patient's body in which the wound 40 was formed. A 0.48 cm hole 44 was drilled in the bottom of the simulated wound bed for vacuum attachment. As shown in FIG. 3, an open-celled polyurethane foam 46 (GranuFoam™, KCI Inc., San Antonio, Tex.) was used as a manifold and placed in the simulated wound bed 40. A structured film 48 (HDPE 21002, emboss #124, 50 micron, 83 mm, Huhtamaki Inc., De Soto, Kans.) was attached to the top surface of the polycarbonate block with adhesive 50 to mimic a rough, skin-like surface. The polycarbonate block 42 was heated to 35° C. for about 10 minutes prior to testing to simulate body temperature.

A two-part sealant sample was mixed and then applied around the wound bed as described in Example 1. The sealant bead 52 was about 7 cm in diameter. The block 42 with the sealant 52 was then allowed to cure for two minutes at 35° C.

A Simplace™ drape (KCI Inc., San Antonio, Tex.) functioning as a sealing member 54 was then placed over the sealant 52 and secured with two sets of five passes of a 4.5 lb rubber roller (95 mm diameter, 45 mm wide); one set perpendicular to the other. A vacuum pump 56 (ActiV.A.C.™ model 60095, KCI Inc., San Antonio, Tex.), which served as the negative pressure source, was connected to the wound bed 40. The time necessary to achieve 125 mm Hg was measured.

Table 4 demonstrates the ability of several Example formulations to seal the sealing member 54 to the structured film 48 located on top of the polycarbonate block 42.

### Peel

A sealant bead of Example 3 was dispensed on several surfaces and allowed to stand for 1 day at room temperature. Table 5 demonstrates that the sealant bead was cleanly removed from each surface.

### EXAMPLES

### Part A

Polyisoprene (70 parts) and polyisobutylene diluent (30 parts) were mixed with a mechanical stirrer (IKA™ RW16 Basic, IKA Works, Inc., Wilmington, Del.) until homogeneous. Pt catalyst (0.7 parts) was added and mixed with the mechanical stirrer. This is Part A.

### Part B

Polyisoprene (70 parts) and polyisobutylene diluent (30 parts) were mixed with the mechanical stirrer until homogeneous. TMCTS cross-linker (3.5 parts) was added and mixed with the mechanical stirrer. This is Part B.

### Example 1

Example 1 (E-1) was prepared by loading approximately 20 mL each of Part A and Part B into a 50 mL cartridge (MixPac #0610441804, Sulzer Mixpac Ltd. Salem, N.H.) loaded into a cartridge dispenser (MixPac #0610441824, Sulzer Mixpac Ltd., Salem, N.H.) equipped with a VPS mixing tip (#70201033167, 3M Company, St. Paul, Minn.). A bead of sealant (mixed Part A and Part B) was dispensed on a glass slide at room temperature and placed in a 35° C. oven to cure. Examples E-2 through E-4 were prepared as E-1 with the formulations shown in Table 2.

### Comparatives

C-1 through C-6 were prepared as E-1 with the formulations shown in Table 2.

TABLE 2
Sealant Formulations

| Part A (parts) | | | Part B (parts) | | |
|---|---|---|---|---|---|
| Crosslinkable | | Cata- | Crosslinkable | | Cross- |
| Polymer | Diluent | lyst | Polymer | Diluent | linker |

EXAMPLES

| | | | | | | |
|---|---|---|---|---|---|---|
| E-1 | 70 (LIR-30) | 30 | 0.7 | 70 (LIR-30) | 30 | 3.5 |
| E-2 | 70 (LIR-30) | 30 | 0.7 | 70 (LIR-30) | 30 | 1.4 |
| E-3 | 30 (LIR-50) | 70 | 0.7 | 30 (LIR-50) | 70 | 3.5 |
| E-4 | 20 (LIR-50) | 80 | 0.7 | 20 (LIR-50) | 80 | 3.5 |

COMPARATIVES

| | | | | | | |
|---|---|---|---|---|---|---|
| C-1 | 10 (LIR-50) | 90 | 0.7 | 20 (LIR-50) | 80 | 3.5 |
| C-2 | 100 | 0 | 1.5 | 100 | 0 | 5 |
| C-3 | 100 (LBR307) | 0 | 1.5 | 100 (LBR307) | 0 | 5 |
| C-4 | 100 | 0 | 1.5 | 100 | 0 | 5 |
| C-5 | 100 | 0 | 1.5 | 100 | 0 | 5 |
| C-6 | 100 (LBR305) | 0 | 1.5 | 100 (LBR305) | 0 | 5 |

### Results

Example and Comparative results for cure, hardness, tack and viscosity are shown in Table 3.

TABLE 3
Results

| Samples | Curing Time | Hardness | Tack | Viscosity (cP) |
|---|---|---|---|---|
| E-1 | 10 min | 35 | Low | 81000 ± 1000 |
| E-2 | 10 min | 21 | High | 81,000 ± 1,000 |
| E-3 | 10 min | 24 | High | 100,000 ± 1,0000 |
| E-4 | 10 min | 21 | High | 47,500 ± 1,500 |
| C-1 | 45 min | 12 | High | 25,500 ± 1,500 |
| C-2 | >3 hours | 30 | Low | 16,000 ± 500 |
| C-3 | >3 hours | 11 | Low | 2,700 ± 1,000 |
| C-4 | >24 hours | Not fully | Not fully | 1,050 ± 500 |
| | | cured | cured | |
| C-5 | >4 hours | 25 | Med | 3,350 ± 150 |
| C-6 | 40 min | 45 | Low | 66,500 ± 500 |

TABLE 4
Sample Sealing Ability

| Samples | Time to Reach 125 mm Hg (sec) |
|---|---|
| E-2 | 2.0 |
| E-3 | 2.6 |
| E-4 | 2.5 |
| Simplace ™ (no sealant) | >30 |

TABLE 5
Removal of Example 3

| Surface | Comments |
|---|---|
| glass | easy and clean removal; no residue |
| aluminum | easy and clean removal; no residue |
| vinyl leather | easy and clean removal; no residue |

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure.

All references and publications cited herein are expressly incorporated herein by reference in their entirety into this disclosure.

Various features and aspects of the present disclosure are set forth in the following claims.

1. A medical sealant composition comprising:
an unsaturated rubber hydrocarbon having at least one hydrosilylation-crosslinkable functional group and a crosslinking agent having at least one Sill group per molecule, wherein the composition is curable at 35 degrees C. in less than 20 minutes,
wherein the composition has a first (uncured) state having a viscosity in the range of 15,000 cP to 1,000,000 cP.

2. The medical sealant composition of claim 1, wherein the composition is curable at 35 degrees C. in less than 15 minutes.

3. The medical sealant composition of claim 1, wherein the composition is curable at 35 degrees C. in less than 10 minutes.

4. A negative pressure wound therapy system comprising the medical sealant composition of claim 1.

5. A method for coupling a medical article to skin, the method comprising:
providing a medical article;
providing a composition comprising an unsaturated rubber hydrocarbon having at least one hydrosilylation-crosslinkable functional group and a crosslinking agent having on the average at least one Sill group per molecule;
applying the composition to one or both of the medical article and skin when the composition is in an uncured state;
applying the medical article to the skin; and
allowing the composition to cure to form a sealant between the medical article and the skin.

6. The method of claim 5, wherein applying the sealant includes dispensing the sealant from a dual-cartridge automix delivery system.

7. The method of claim 5, wherein the medical article is a component of a negative pressure wound therapy system wherein providing a medical article includes providing the component; wherein applying the composition to one or both of the medical article and skin includes applying the composition to one or both of the component and the skin; wherein applying the medical article to the skin after applying the composition includes applying the component to the skin.

8. The method of claim 5, wherein the composition has a first (uncured) state having a viscosity of at least 15,000 cP.

9. (canceled)

10. (canceled)

11. The method of claim 5, wherein the composition forms a second state having a shore-hardness ranging from about 10 to about 50, after curing.

12. The method of claim 5, wherein the composition forms a second state having a shore-hardness ranging from about 15 to about 40, after curing.

13. The method of claim 5, wherein the composition forms a second state having a shore-hardness ranging from about 15 to about 35, after curing.

14. The medical sealant composition of claim 1, wherein the unsaturated rubber hydrocarbon comprises polyisoprene.

15. The medical sealant composition of claim 14, wherein the polyisoprene has a molecular weight ranging from about 10000 to about 90000.

16. The medical sealant composition of claim 1, wherein the composition further comprises a polymer diluent.

17. The medical sealant composition of claim 16, wherein the polymer diluents includes an unreactive rubber, mineral oil, or a combination thereof.

18. The medical sealant composition of claim 16, wherein the polymer diluent comprises polyisobutylene.

19. The medical sealant composition of claim 1, wherein the composition further comprises a catalyst.

20. The medical sealant composition of claim 1, wherein the composition is a two-part system comprising a first part comprising the unsaturated rubber hydrocarbon and a second part comprising the unsaturated rubber hydrocarbon and the crosslinking agent.

21. The medical sealant composition of claim 20, wherein the first part further comprises a catalyst.

22. The medical sealant composition of claim 20, wherein the first part and the second part of the two-part system are kept separate prior to use.
